(19) United States
(12) Patent Application Publication
(10) Pub. No.: US 2012/0271071 A1
(43) Pub. Date: Oct. 25, 2012

(54) TRANSALKYLATION OF METHYLATED AROMATIC HYDROCARBON-ENRICHED FRACTIONS IN C8 AROMATIC HYDROCARBON PRODUCTION

(75) Inventors: ROBERT HAIZMANN, ROLLING MEADOWS, IL (US); PAUL ALVIN SECHRIST, SOUTH BARRINGTON, IL (US)

(73) Assignee: UOP LLC, DES PLAINES, IL (US)

(21) Appl. No.: 13/092,289

(22) Filed: Apr. 22, 2011

(51) Int. Cl. C07C 15/067

(52) U.S. Cl. 585/1

(57) ABSTRACT

Methods are disclosed for producing C₈ aromatic hydrocarbons. Representative methods comprise (a) fractionating an aromatic hydrocarbon containing feed stream (e.g., a feed stream comprising C₉ and/or C₁₀ aromatic hydrocarbons), to provide at least one methylated aromatic hydrocarbon-enriched fraction; and (b) reacting at least a portion of the at least one methylated aromatic hydrocarbon-enriched fraction in a transalkylation reaction zone to provide a transalkylation effluent comprising the C₈ aromatic hydrocarbons. The presence of a methylated aromatic hydrocarbon-enriched fraction in the inlet stream to the transalkylation reaction zone provides a number of advantages as described herein.

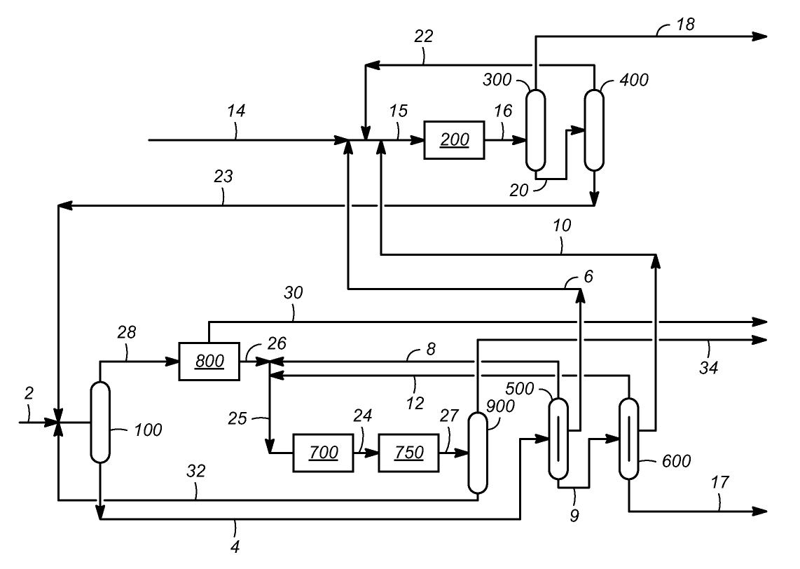

### FIELD OF THE INVENTION

The present invention relates to methods for producing C₈ aromatic hydrocarbons by transalkylating a fraction that is enriched in methylated aromatic hydrocarbons (e.g., enriched in trimethylbenzene as a C₉ aromatic hydrocarbon or enriched in tetramethylbenzene as a C₁₀ aromatic hydrocarbon), for example by fractionation of a feed stream prior to a transalkylation reaction zone. The methods are particularly applicable in an overall aromatics complex for the production of para-xylene from a product of crude oil refining (e.g., reformate obtained from naphtha reforming).

### DESCRIPTION OF RELATED ART

The isomers of xylene (dimethylbenzene), namely ortho-xylene, meta-xylene, and para-xylene, are important chemical intermediates, with para-xylene having by far the greatest commercial significance. The primary commercial application of para-xylene involves its oxidation to make terephthalic acid. Terephthalic acid, in turn, is used to make polymers such as polytrimethyleneterephthalate (PTT), polybutyleneterephthalate (PBT), and polyethyleneterephthalate (PET). PET, one of the largest volume polymers in the world, is made via condensation polymerization of terephthalic acid with ethylene glycol. Given the large market for PET plastics and fibers, in addition to other end products produced from para-xylene, there is a substantial demand for this intermediate in high purity. This demand in fact outweighs the yield of para-xylene, as derived from conventional crude oil refining processes such as catalytic reforming to produce aromatic hydrocarbons.

Reforming generally refers to the conversion (or “aromatization”) of a naphtha hydrocarbon feed, as a crude oil fraction, to the major products of benzene, toluene, and the xylene isomers. The effluent of the reforming reaction zone or reformer (e.g., a catalytic reformer with continuous catalyst regeneration) is separated by distillation and in particular using a reformate splitter such that the C₆ and C₇ aromatic hydrocarbons, namely benzene and toluene, are often recovered in a splitter overhead fraction, and the C₈ aromatic hydrocarbons, namely the xylene isomers and ethylbenzene, are essentially all recovered in a splitter bottoms fraction at approximately their equilibrium concentrations. Higher-boiling C₉ and C₁₀ aromatic hydrocarbons contained in this reformate splitter bottoms fraction are conventionally separated by distillation, for example using a xylene splitter, from the C₈ aromatic hydrocarbons in this stream. The desired para-xylene can then be separated from the mixture of C₈ aromatic hydrocarbons contained in the xylene splitter overhead fraction, using a para-xylene separation process. Due to the similar boiling points of the xylene isomers, xylene separation processes are not performed economically using distillation. Such processes instead generally rely on adsorptive separation using a simulated moving bed (SMB) of adsorbent having micropores of the proper size and geometry for selectively adsorbing para-xylene over the other xylene isomers. Representative adsorbents and processes for the selective separation para-xylene in this manner are described, for example, in U.S. Pat. No. 3,686,342, U.S. Pat. No. 3,903,187, U.S. Pat. No. 4,313,015, U.S. Pat. No. 4,899,017, U.S. Pat. No. 5,171,922, U.S. Pat. No. 5,177,295, U.S. Pat. No. 5,495,061, and U.S. Pat. No. 5,948,950.

Downstream of the xylene separation zone, a para-xylene-depleted raffinate, comprising predominantly the other C₈ aromatic hydrocarbons, ortho-xylene and meta-xylene, is typically further processed in an isomerization reaction zone to restore approximately an equilibrium concentration of the xylene isomers, including about 20-25% by weight of para-xylene. The para-xylene produced from isomerization may advantageously be recycled to the xylene separation zone for its separation and recovery, thereby improving the overall yield of para-xylene, while recycling the other xylene isomers essentially to extinction. Adsorptive separation and isomerization reaction zones, operating in combination with the recycle of less desirable xylene isomers, are therefore commercially effective for the substantially complete production and recovery of para-xylene from the overall C₈ aromatic hydrocarbons obtained from catalytic reforming.

Depending on the catalyst used in the isomerization reaction zone, ethylbenzene is also isomerized at least to some extent to produce mixed xylenes, from which additional para-xylene is ultimately produced and separated, with the same high efficiency, in the xylene isomerization/separation loop. Isomerization thereby prevents the excessive accumulation of ethylbenzene, which co-boils with the xylene isomers and is therefore not economically separated by distillation, in this loop. Isomerization catalysts may also have a dealkylation functionality, whereby benzene is formed as a desired product, or, depending on demand, further reacted to obtain additional xylenes by transalkylation, as discussed below.

In addition to the use of adsorptive separation and isomerization in the overall production of para-xylene from C₈ aromatic hydrocarbons, other processes may be incorporated into an overall aromatics complex with the objective of converting aromatic hydrocarbons having different carbon numbers to additional xylenes that may be recovered as para-xylene, as discussed above. An important process in this regard involves disproportionation and/or transalkylation reactions, such as the conversion of two molecules of the C₇ aromatic hydrocarbon, toluene, to benzene and xylene (i.e., by disproportionation) or the conversion of toluene and a C₉ aromatic hydrocarbon such as trimethylbenzene, to two xylene molecules (i.e., by transalkylation). Like the C₈ aromatic hydrocarbons, the C₆, C₇, C₉, and even C₁₀ aromatic hydrocarbons are in general also obtained as products of the upstream catalytic reforming reactions. As discussed above, for example, C₆ and C₇ aromatic hydrocarbons (benzene and toluene), are normally concentrated into the reformate splitter overhead fraction. Thereafter, these aromatic hydrocarbons are extracted and treated to remove olefins. The C₉ and C₁₀ aromatic hydrocarbons from reforming are present, together with the C₈ aromatics, in the reformate splitter bottoms. These higher carbon number aromatic hydrocarbons are then removed in a bottoms fraction of the xylene splitter, which separates the C₈ aromatics into the overhead fraction.

In general, the production of para-xylene is practiced commercially in large-scale facilities and is highly competitive. Petrochemical producers must strive continually to achieve the highest possible performance characteristics (e.g., conversion, separation, and recovery) in integrated process units, in the most economically attractive manner (e.g., in terms of capital and operating costs). To this end, a conventional aromatics complex is described in Meyers et al., HANDBOOK OF PETROLEUM REFINING PROCESSES (part 2), 2d. Edition, in 1997 published by McGraw-Hill. Flow schemes and other considerations associated with various process steps involved in aromatics complexes are described, for example, in U.S. Pat. No. 4,341,914; U.S. Pat. No. 6,512,154; U.S. Pat. No. 6,740,788; U.S. Pat. No. 6,774,273; U.S. Pat. No. 6,867,339; and US 2004/0186332.

The role of the above-described disproportionation and transalkylation reactions in an overall aromatics production complex is very significant in terms of converting aromatic hydrocarbons, differing in carbon number from xylenes and ethylbenzene, to additional C₈ aromatic hydrocarbons. This increases the overall yield of xylenes and ethylbenzene, which are in turn introduced into the xylene separation/isomerization loop, in order to separate para-xylene with high efficiency and produce additional amounts of this desired isomer, by isomerizing the mixture toward equilibrium isomer concentrations. Improvements associated with the transalkylation reaction zone are therefore of great industrial importance in the overall objective of advancing aromatics complex processing technology.

### SUMMARY OF THE INVENTION

The present invention is associated with the discovery of methods for producing C₈ aromatic hydrocarbons by the transalkylation of non-C₈ aromatic hydrocarbons, for example C₇ and C₉ aromatic hydrocarbons. Transalkylation refers to any of a number of reactions that result in (1) one molecule, introduced into a transalkylation reaction zone (or contacted with a transalkylation catalyst) gaining an alkyl group and (2) another molecule, introduced into the transalkylation reaction zone, losing an alkyl group. An exemplary reaction occurring in a transalkylation reaction zone is therefore the conversion of toluene and trimethylbenzene to two molecules of xylene, according to the reaction

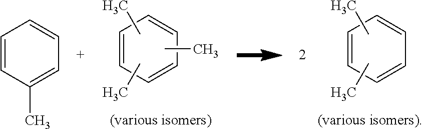

Advantageously, according to embodiments of the invention, the inlet stream (e.g., a transalkylation combined feed) to the transalkylation reaction zone comprises some or all (i.e., at least a portion) of a methylated aromatic hydrocarbon-enriched fraction. This fraction may be obtained from fractionating (distilling) an aromatic hydrocarbon containing feed stream, for example a feed stream comprising C₉ or C₁₀ aromatic hydrocarbons.

The presence of a methylated aromatic hydrocarbon-enriched fraction in the inlet stream to the transalkylation reaction zone provides important advantages with respect to improving the performance in this zone. In particular, aspects of the invention are associated with the finding that transalkylation processes are significantly more selective for transalkylating methyl group substituents of an aromatic ring (e.g., a benzene ring), compared to higher alkyl group substituents, including ethyl, propyl, and butyl substituents. In fact, these higher alkyl groups are much more susceptible, under transalkylation conditions, to being non-selectively dealkylated or removed from the aromatic ring altogether, thereby forming benzene and light alkane hydrocarbons, having significantly less value. Further aspects of the invention are associated with the finding that crude oil refining processes (e.g., catalytic reforming), which are often used as a source of alkylated aromatic hydrocarbons in an aromatics complex, generally produce methylated aromatic hydrocarbons (e.g., aromatic hydrocarbons substituted with one or more methyl groups but not higher alkyl groups) at a concentration well below their concentrations at equilibrium in a mixture of ethyl-, propyl-, and/or butyl-substituted aromatic hydrocarbons of the same carbon number. Therefore, in the conventional use of fractions of catalytic reforming effluent (or reformate) that are not enriched in methylated aromatic hydrocarbons, a high proportion of the alkylated aromatic hydrocarbons are dealkylated in the transalkylation reaction zone, limiting the production of the desired C₈ aromatic hydrocarbons and detrimentally increasing the yield of lower value byproducts such as the light alkane hydrocarbons, ethane, propane, and butanes.

Embodiments of the invention are therefore directed to methods for producing C₈ aromatic hydrocarbons. Representative methods comprise (a) fractionating an aromatic hydrocarbon containing feed stream (e.g., a feed stream comprising C₉ or C₁₀ aromatic hydrocarbons), to provide at least one methylated aromatic hydrocarbon-enriched fraction; and (b) reacting at least a portion of the at least one methylated aromatic hydrocarbon-enriched fraction in a transalkylation reaction zone to provide a transalkylation effluent comprising the C₈ aromatic hydrocarbons.

Other embodiments of the invention are directed to methods for producing C₈ aromatic hydrocarbons, comprising: (a) isomerizing, in an isomerization reaction zone, a methylated aromatic hydrocarbon-depleted fraction to provide an isomerization effluent comprising an additional amount (i.e., the net amount produced from isomerization) of methylated aromatic hydrocarbons; (b) fractionating the isomerization effluent to provide at least one methylated aromatic hydrocarbon-enriched fraction; and (c) reacting at least a portion of the at least one methylated aromatic hydrocarbon-enriched fraction in a transalkylation reaction zone to provide a transalkylation effluent comprising the C₈ aromatic hydrocarbons.

Yet further embodiments of the invention are directed to methods for producing C₈ aromatic hydrocarbons, comprising reacting a methylated aromatic hydrocarbon (e.g., trimethylbenzene and/or tetramethylbenzene) in a transalkylation reaction zone to provide a transalkylation effluent comprising the C₈ aromatic hydrocarbons, wherein the methylated aromatic hydrocarbon is present in a methylated aromatic hydrocarbon-enriched fraction that is fed to the transalkylation reaction zone.

These and other embodiments and aspects relating to the present invention are apparent from the following Detailed Description.

### BRIEF DESCRIPTION OF THE DRAWING

The FIGURE depicts a representative process that may be used in an aromatics complex for producing C₈ aromatic hydrocarbons and particularly para-xylene.

The features referred to in the FIGURE are not necessarily drawn to scale and should be understood to present an illustration of a representative embodiment of the invention and/or principles involved. Processes according to other embodiments of the invention will have configurations, components, and operating parameters determined, in part, by their intended application and the environment in which they are used.

### DETAILED DESCRIPTION

The feed stream, according to aspects of the invention, is any aromatic hydrocarbon containing stream, and preferably comprises C₉ or C₁₀ aromatic hydrocarbons. Representative feed streams include fractions of reformate (i.e., catalytic reforming effluent). Such fractions may be obtained, for example, from the catalytic reforming of naphtha, followed by separation of the reforming effluent using a reformate splitter, and further separation of a first high boiling (e.g., bottoms) product exiting the reformate splitter using a xylene column to recover a second high boiling (e.g., bottoms) product exiting the xylene column. In this case, the first high boiling product often contains predominantly (e.g., greater than 50%, and often greater than about 80%, by weight) C₈ and higher carbon number aromatic hydrocarbons, and the second high boiling product, which may be used as a feed stream in representative embodiments of the invention, often contains predominantly (e.g., greater than 50%, and often greater than about 80%, by weight) C₉ and higher carbon number aromatic hydrocarbons.

Representative methods comprise fractionating the feed stream to provide a methylated aromatic hydrocarbon-enriched fraction. The term “methylated aromatic hydrocarbon-enriched fraction” refers to a fraction of the feed stream that contains a higher percentage by weight of methylated aromatic hydrocarbons, based on aromatic hydrocarbons of the same carbon number, relative to the feed stream. A “methylated aromatic hydrocarbon” is an aromatic ring-containing hydrocarbon (e.g., a benzene-ring containing hydrocarbon) in which the carbon atoms of the aromatic ring are either unsubstituted or substituted with a methyl group. To illustrate, a fraction containing 80% by weight of trimethylbenzene and 90% by weight of C₉ aromatic hydrocarbons (i.e., 89% by weight of all C₉ aromatic hydrocarbons being trimethylbenzene) would be a “trimethylbenzene-enriched fraction” in the case of a feed stream, being fractionated to obtain such a fraction, containing 50% by weight of trimethylbenzene and 90% by weight of C₉ aromatic hydrocarbons (i.e., 56% by weight of all C₉ aromatic hydrocarbons of the feed stream being trimethylbenzene).

Therefore, a representative methylated aromatic hydrocarbon-enriched fraction has a higher percentage by weight of (i.e., is enriched in) trimethylbenzene, based on all C₉ aromatic hydrocarbons in the aromatic hydrocarbon-enriched fraction, relative to the percentage by weight of trimethylbenzene in the feed stream, based on all C₉ aromatic hydrocarbons in the feed stream. The C₉ aromatic hydrocarbons in the methylated aromatic hydrocarbon-enriched fraction, as well as in the feed stream, include C₉ aromatic hydrocarbons other than trimethylbenzene, namely ethylmethylbenzene (various isomers) and propylbenzene (both normal- and iso-propyl benzene). Another representative methylated aromatic hydrocarbon-enriched fraction has a higher percentage by weight of (i.e., is enriched in) tetramethylbenzene, based on all C₁₀ aromatic hydrocarbons in the aromatic hydrocarbon-enriched fraction, relative to the percentage by weight of tetramethylbenzene in the feed stream, based on all C₁₀ aromatic hydrocarbons in the feed stream. The C₁₀ aromatic hydrocarbons in the methylated aromatic hydrocarbon-enriched fraction, as well as in the feed stream, include C₁₀ aromatic hydrocarbons other than tetramethylbenzene, namely indane and various isomers of dimethylethylbenzene, ethylpropylbenzene, and diethylbenzene, as well as butylbenzene (having both straight-chained and branched-chained butyl groups).

A particular example of a methylated aromatic hydrocarbon-enriched fraction is a trimethylbenzene-enriched fraction obtained from fractionating an aromatic hydrocarbon stream (as an example of a feed stream) comprising C₉ aromatic hydrocarbons (which may comprise both C₉ and C₁₀ aromatic hydrocarbons). Another example of a methylated aromatic hydrocarbon-enriched fraction is a tetramethylbenzene-enriched fraction obtained from fractionating an aromatic hydrocarbon-containing stream comprising C₁₀ aromatic hydrocarbons (as another example of a feed stream). According to representative embodiments, the methylated aromatic hydrocarbon-enriched fraction generally comprises predominantly (e.g., greater than about 50% by weight) methylated aromatic hydrocarbons. Typically, this fraction comprises greater than about 75% by weight, and often greater than about 90% by weight, of methylated aromatic hydrocarbons.

A representative feed stream, comprising C₉ and/or C₁₀ aromatic hydrocarbons, is a feed stream to a fractionation column that is used to provide the methylated aromatic hydrocarbon-enriched fraction. Representative apparatuses for fractionating the feed stream include distillation columns. The methylated aromatic hydrocarbon-enriched fraction is therefore generally obtained as a “cut” or product (e.g., a bottoms product, an overhead product, or a side cut product) of a distillation column that may be used in combination with one or more other distillation columns for fractionating the feed stream. Often, only a single distillation column is used to provide the methylated aromatic hydrocarbon-enriched fraction. According to a particular embodiment, a trimethylbenzene-enriched fraction or a tetramethylbenzene-enriched fraction may be obtained as a side cut product of a distillation column for fractionating the feed stream, for example the second high boiling product exiting the xylene column, as described above. In general, the recovery of the methylated aromatic hydrocarbon-enriched fraction as a side cut product often represents a convenient mode of operating a distillation column used to obtain bottoms and overhead products comprising substantially all (e.g., 95% by weight or more) aromatic hydrocarbons having higher and lower carbon numbers, respectively.

For example, a distillation column used for distilling all or substantially all (e.g., 99% by weight or more) C₉ aromatic hydrocarbons of the feed stream out of the bottoms product and into overhead and side cut products, may be designated an “A9” distillation column. In general, the bottoms product of an A9 distillation column therefore comprises substantially all (e.g., 95% by weight or more) aromatic hydrocarbons having 10 and higher numbers of carbon atoms. This bottoms product may therefore be referred to as a C₁₀ aromatic hydrocarbon-enriched fraction, as this bottoms product has a higher percentage by weight of C₁₀ aromatic hydrocarbons than the feed stream (in this case the feed to the A9 distillation column). In general, the characterization of a fraction as being enriched in an aromatic hydrocarbon of a given carbon number (e.g., a “C₁₀ aromatic hydrocarbon-enriched fraction”) refers to a fraction of a feed stream that contains a higher percentage by weight of aromatic hydrocarbons, based on all aromatic hydrocarbons, relative to the feed stream.

Because, with respect to C₉ aromatic hydrocarbons, trimethylbenzene has a higher boiling point compared to isopropylbenzene (cumene) and other C₉ alkylated benzenes, the trimethylbenzene-enriched fraction may be obtained as a side cut product of the A9 distillation column The overhead product of the A9 distillation column may consequently be enriched in other isomers of C₉ alkylated benzenes, and may be referred to as a trimethylbenzene-depleted fraction.

Likewise, a distillation column used for distilling all or substantially all (e.g., 99% by weight or more) C₁₀ aromatic hydrocarbons of the feed stream out of the bottoms product and into overhead and side cut products, may be designated an “A10” distillation column. In general, the bottoms product of an A10 distillation column therefore comprises substantially all (e.g., 95% by weight or more) aromatic hydrocarbons having 11 and higher numbers of carbon atoms. This bottoms product may therefore be referred to as a C₁₁ aromatic hydrocarbon-enriched fraction, as this bottoms product has a higher percentage by weight of C₁₁ aromatic hydrocarbons than the feed stream (in this case the feed to the A10 distillation column). This bottoms product is often a heavy aromatic hydrocarbon drag stream, used to prevent any substantial accumulation of heavy aromatic hydrocarbons in recycle loops of an aromatics complex. Because, with respect to C₁₀ aromatic hydrocarbons, tetramethylbenzene has a higher boiling point compared to other C₁₀ alkylated benzenes, the tetramethylbenzene-enriched fraction may be obtained as a side cut product of the A10 distillation column. The overhead product of the A10 distillation column may consequently be enriched in other isomers of C₁₀ alkylated benzenes, and may be referred to as a tetramethylbenzene-depleted fraction. Representative A9 and/or A10 distillation columns are divided wall distillation columns A divided wall distillation column is understood in the art as referring to a two-column Petyluk distillation arrangement, placed within a common vessel, but separated into parallel, elongated sections using a wall extending vertically within the column over a substantial portion of its axial length. Combining columns in a single shell allows for significant gains in energy efficiency, due to the sharing of bottoms reboiler and overhead condenser duties. Divided wall distillation columns are described, for example, in U.S. Pat. No. 2,471,134; U.S. Pat. No. 4,230,533; and U.S. Pat. No. 6,551,465.

According to representative embodiments, therefore, the trimethylbenzene-enriched fraction may be obtained as a side cut product of an A9 distillation column for fractionating a feed stream comprising C₉ aromatic hydrocarbons (e.g., a feed stream comprising both C₉ and C₁₀ aromatic hydrocarbons). Such embodiments may further comprise fractionating the C₁₀ aromatic hydrocarbon-enriched fraction of the A9 distillation column (e.g., as the bottoms product of this column) to provide a tetramethylbenzene-enriched fraction, for example as a side cut product of an A10 distillation column. In general, therefore, the tetramethylbenzene-enriched fraction may be obtained as a side cut product of an A10 distillation column for fractionating a feed stream comprising C₁₀ aromatic hydrocarbons.

Methods according to embodiments of the invention comprise, in addition to providing a methylated aromatic hydrocarbon-enriched fraction, reacting this fraction in a transalkylation reaction zone to provide a transalkylation effluent comprising C₈ aromatic hydrocarbons. As discussed above, important benefits are obtained from the use of a methylated aromatic hydrocarbon-enriched fraction, including a decrease in non-selective, dealkylated reaction products (e.g., light alkane hydrocarbons and benzene) and an increase in the desired xylene reaction products, which result from transalkylation. In addition to transalkylation reactions (e.g., the reaction of toluene and trimethylbenzene molecules to produce two xylene molecules as described above) the transalkylation reaction zone is understood to optionally cause disproportionation reactions as well, with the most common and desired of these being the reaction of two molecules of toluene to produce benzene and xylene molecules, thereby adding to the total yield of C₈ aromatic hydrocarbons. In general, both transalkylation and disproportionation reactions proceed directionally toward an equilibrium distribution of benzene and alkylated aromatic hydrocarbons.

The transalkylation reaction zone generally comprises a catalyst, and is maintained under transalkylation conditions, known for carrying out the desired transalkylation and optionally disproportionation reactions. Generally, both types of reactions occur, and the extent to which one type of reaction occurs relative to another type is governed primarily by the composition of the transalkylation combined feed (e.g., the total of all streams entering the transalkylation reaction zone). For example, a higher concentration of toluene in the transalkylation combined feed directionally leads to a greater extent of toluene disproportionation to benzene and xylene.

Representative transalkylation conditions include a temperature generally from about 100° C. (212° F.) to about 425° C. (797° F.), and typically from about 200° C. (392° F.) to about 400° C. (752° F.). In commercial operation, the average transalkylation reaction zone temperature is often increased over a period of operation, in order to compensate for gradually decreasing activity of the catalyst. Other representative transalkylation conditions include an absolute pressure generally from about 100 kPa (14.5 psi) to about 10 MPa (1450 psi), and typically from about 0.5 MPa (72.5 psi) to about 5 MPa (725 psi). Further transalkylation conditions include a weight hourly space velocity (WHSV, based on the transalkylation combined feed) generally from about 0.1 hr⁻¹ to about 30 hr⁻¹, typically from about 0.5 hr⁻¹ to about 20 hr⁻¹, and often from about 1 hr⁻¹ to about 5 hr⁻¹. As is understood in the art, the WHSV is the weight flow of the liquid charged to the reactor (e.g., the transalkylation combined feed) divided by the weight of the catalyst bed and represents the equivalent catalyst bed weights of feed processed every hour. The WHSV is related to the inverse of the reactor residence time.

Representative transalkylation catalysts used in the transalkylation reaction zone comprise a metal component and an acidic component, such as an acidic molecular sieve that may be zeolitic or non-zeolitic. A transalkylation catalyst may comprise the metal component and the acidic molecular sieve component, in addition to an inorganic oxide component. Typically, the metal component has hydrogenation functionality, and may comprise at least one noble metal and at least one base metal. Representative noble metals include platinum group metals selected from the group consisting of platinum, palladium, rhodium, ruthenium, osmium, iridium and mixtures thereof. Representative base metals are selected from the group consisting of rhenium, tin, germanium, lead, iron, cobalt, nickel, indium, gallium, zinc, uranium, dysprosium, thallium, and mixtures thereof. A promoter or modifier metal may also be used in the transalkylation catalyst, and particular examples of promoters or modifiers include metals in IUPAC groups 1, 2, 5, 6, 7, 11, 12, 13, 14, 15, 16 and 17. According to a particular embodiment, the metal component of the transalkylation catalyst comprises platinum in combination with tin and/or rhenium. The total content of metal(s) in the transalkylation catalyst is generally from about 0.01% to about 10% by weight, and typically from about 0.01% to about 3% by weight. The total content of the acidic molecular sieve in the transalkylation catalyst is generally from about 1% to about 99% by weight, typically from about 10% to about 90% by weight, and often from about 25% to about 75% by weight. Additional components of the transalkylation catalyst may include the inorganic oxide component, such as binder material (e.g., alumina).

The transalkylation reaction zone may comprise one or more individual transalkylation reactors containing transalkylation catalyst in various catalyst bed configurations (e.g., fixed bed or moving bed) and flow configurations (e.g., axial flow or radial flow). Each of the one or more individual reactors may contain one or more types of transalkylation catalyst. If two or more types are used, they may be blended, at varying blend ratios, or segregated in the individual reactor(s).

A representative, non-limiting embodiment of the invention, as it may be practiced in the context of various integrated processes in an overall aromatics complex for producing para-xylene in addition to other products, is illustrated in the FIGURE. A representative feed stream 4 comprises C₉ and C₁₀ aromatic hydrocarbons and is obtained as a bottoms product (e.g., a second high boiling product as discussed above) of xylene column 100. Xylene column 100 is used to separate, in addition to other steams, a first high boiling product, for example bottoms product 2 of a reformate splitter (not shown), as discussed above. Methods according to the embodiment shown in the FIGURE therefore include fractionating feed stream 4 to provide at least three fractions, namely trimethylbenzene-enriched fraction 6 as a medium boiling fraction, trimethylbenzene-depleted fraction 8 as a low boiling fraction, and C₁₀ aromatic hydrocarbon-enriched fraction 9 as a high boiling fraction. The designation of fractions as “low boiling,” “medium boiling,” or “high boiling,” as obtained from a particular fractionation column, refers to the relative normal boiling point ranges of these fractions. In the embodiment shown in the FIGURE, a subsequent fractionation comprises fractionating C₁₀ aromatic hydrocarbon-enriched fraction 9 to provide at least three additional fractions, namely tetramethylbenzene-enriched fraction 10 as a medium boiling fraction, tetramethylbenzene-depleted fraction 12 as a low boiling fraction, and heavy aromatics fraction 17, which is generally enriched in C₁₁ aromatic hydrocarbons and even higher carbon number aromatic hydrocarbons, and serves as a heavy aromatic hydrocarbon drag stream, as discussed above. In the embodiment shown in the FIGURE, C₁₀ aromatic hydrocarbon-enriched fraction 9 is therefore fractionated to provide tetramethylbenzene-enriched fraction 10 as a methylated aromatic hydrocarbon-enriched fraction. Thus, C₁₀ aromatic hydrocarbon-enriched fraction 9 may also be considered a feed stream comprising C₁₀ aromatic hydrocarbons that is a source of a methylated aromatic hydrocarbon-enriched fraction, following fractionation.

Fractionation columns used in the fractionations described above, for (i) distilling all or substantially all (e.g., 99% by weight or more) C₉ aromatic hydrocarbons of the feed stream out of the bottoms product (or C₁₀ aromatic hydrocarbon-enriched fraction), and (ii) distilling all or substantially all (e.g., 99% by weight or more) C₁₀ aromatic hydrocarbons of the feed stream out of the bottoms product (or heavy aromatics fraction, enriched in C₁₁ aromatic hydrocarbons), may be referred to as “A9” and “A10” distillation columns, respectively. In the embodiment shown in the FIGURE, the separations of feed streams comprising C₉ and/or C₁₀ aromatic hydrocarbons may be performed using A9 divided wall distillation column 500 and A10 divided wall distillation column 600, respectively.

In the embodiment shown in the FIGURE, both methylated aromatic hydrocarbon-enriched fractions, namely trimethylbenzene-enriched fraction 6 and tetramethylbenzene-enriched fraction 10 (as side cut products of A9 divided wall distillation column 500 and A10 divided wall distillation column 600, respectively), are reacted in transalkylation reaction zone 200. In this case, these methylated aromatic hydrocarbon-enriched fractions 6, 10 are reacted together with (i) recycled, toluene-enriched product 22 and (ii) benzene and/or toluene-enriched fraction 14, which may be obtained as a low boiling product, for example an overhead product, of a reformate splitter (not shown), following the extraction of aromatic hydrocarbons. Preferably, toluene-enriched fraction 14 contains substantially all of the benzene and toluene exiting this reformate splitter, which is generally used to separate products exiting one or more upstream catalytic reforming operations. Particular processes for performing aromatic hydrocarbon extraction utilize sulfolane (i.e., tetrahydrothiophene dioxide, also known as tetramethylene sulfone) as a physical solvent to selectively dissolve aromatic hydrocarbons from various fractions, such as the reformate splitter overhead. See, for example, the UOP Sulfolane® Process as described by Meyers, R. A., “Handbook of Petroleum Refining Processes,” The McGraw-Hill Companies, Inc. (2004), pp. 2.13-2.23. The important benefits of reacting at least one methylated aromatic hydrocarbon-enriched fraction in a transalkylation reaction zone are discussed in detail above.

Transalkylation combined feed 15 is the total of all components passed to transalkylation reaction zone 200, and, in the embodiment shown in the FIGURE, includes toluene-enriched fraction 14, trimethylbenzene-enriched fraction 6, and tetramethylbenzene-enriched fraction 10, in addition to toluene-enriched product 22 (of toluene column 400). Reacting trimethylbenzene-enriched fraction 6 and tetramethylbenzene-enriched fraction 10, as methylated aromatic hydrocarbon-enriched fractions, in transalkylation reaction zone 200 provides transalkylation effluent 16 (exiting transalkylation reaction zone 200). As a result of transalkylation and possibly disproportionation reactions, transalkylation effluent 16 comprises C₈ aromatic hydrocarbons (xylenes), and in particular a net amount of C₈ aromatic hydrocarbons generated in transalkylation reaction zone 200, beyond the amount of C₈ aromatic hydrocarbons introduced in transalkylation combined feed 15. The net amount of C₈ aromatic hydrocarbons generated in transalkylation reaction zone 200, importantly, represents essentially the net production of C₈ aromatic hydrocarbons in the embodiment shown in the FIGURE (and not introduced in bottoms product 2 of a reformate splitter). In addition to these C₈ aromatic hydrocarbons, transalkylation effluent 16 also normally comprises benzene and toluene as unreacted components and/or as reaction products of transalkylation and disproportionation.

Representative methods therefore comprise fractionating the transalkylation effluent to provide a transalkylation product comprising the C₈ aromatic hydrocarbons. In the embodiment shown in the FIGURE, transalkylation effluent 16 is fed to benzene column 300 for the removal, in a low boiling fraction (e.g., an overhead product), of benzene product 18 and possibly light byproducts (e.g., light alkanes) formed in transalkylation reaction zone 200. A high boiling fraction, such as benzene column bottoms product 20 is fed to toluene column 400 for the removal, in a low boiling fraction, of toluene-enriched product 22 that may be partly or completely recycled to transalkylation reaction zone 200 for production of additional benzene and C₈ aromatic hydrocarbons (xylenes). A high boiling fraction (e.g., a bottoms product) of toluene column 400, such as transalkylation product 23, comprises C₈ aromatic hydrocarbons produced in transalkylation reaction zone 200, in addition to C₉ and higher carbon number aromatic hydrocarbons, as unreacted components and/or as reaction products of transalkylation and disproportionation.

Methods according to the particular embodiment of the FIGURE can therefore include fractionating transalkylation effluent 16 in benzene column 300 to provide benzene product 18 (enriched in benzene), as a low boiling fraction (e.g., overhead product) of this column, and benzene column bottoms product 20, as a high boiling fraction (e.g., bottoms product) of this column. These methods can also include fractionating benzene column bottoms product 20 in toluene column 400 to provide toluene-enriched product 22, for example as a toluene-enriched recycle stream, as a low boiling fraction (e.g., overhead product) of this column and transalkylation product 23, as a high boiling fraction (e.g., bottoms product) of this column.

As a result of the fractionations occurring in A9 and A10 divided wall distillation columns 500, 600, trimethylbenzene-depleted fraction 8 and tetramethylbenzene-depleted fraction 12 contain a distribution of alkylated aromatic hydrocarbons that is generally far from an equilibrium distribution. As illustrated in the embodiment shown in the FIGURE, representative methods comprise producing additional amounts of trimethylbenzene and tetramethylbenzene by isomerizing trimethylbenzene-depleted fraction 8 and tetramethylbenzene-depleted fraction 12 in isomerization reaction zone 700 to provide isomerization effluent 24 comprising these additional amounts. Isomerization of these methylated aromatic hydrocarbon-depleted streams 8, 12 can also provide an isomerate product 32 (e.g., a high boiling fraction such as a bottoms product of isomerate splitter 900), following fractionation of isomerization effluent 24, comprising the additional amounts of trimethylbenzene and tetramethylbenzene. The net production of these methylated aromatic hydrocarbons results from the catalyst and conditions used in isomerization reaction zone 700, as described herein, which promote the reactions in this zone toward achieving equilibrium levels of alkylated aromatic hydrocarbons. According to particular embodiments, for example, the yield of trimethylbenzene from converting, in isomerization reaction zone 700, C₉ aromatic hydrocarbons in trimethylbenzene-depleted fraction 8 is generally at least about 50% (e.g., from about 50% to about 95%) and often at least about 60% (e.g., from about 65% to about 90%). According to other particular embodiments, the yield of tetramethylbenzene from converting, in isomerization reaction zone 700, of C₁₀ aromatic hydrocarbons in tetramethylbenzene-depleted fraction 8 is generally at least about 25% (e.g., from about 25% to about 70%) and often at least about 35% (e.g., from about 35% to about 65%). These representative yields are based on a 100% yield being the theoretical yield, for example obtained upon complete conversion of all C₉ aromatic hydrocarbons, other than trimethylbenzene, to trimethylbenzene, or otherwise obtained upon complete conversion of all C₁₀ aromatic hydrocarbons, other than tetramethylbenzene, to tetramethylbenzene.

Isomerization combined feed 25 is the total of all components passed to isomerization reaction zone 700, and, in the embodiment shown in the FIGURE, includes trimethylbenzene-depleted fraction 8, tetramethylbenzene-depleted fraction 12, and para-xylene-depleted raffinate 26 from xylene separation zone 800. As discussed above, xylene separation zone 800 is often an adsorptive separation zone using a simulated moving bed (SMB) of adsorbent, for example comprising a zeolitic molecular sieve, which selectively adsorbs para-xylene over the other xylene isomers. Para-xylene entering this zone, for example in xylene column overhead fraction 28 or other fraction enriched in xylenes, is separated into a para-xylene product, and typically para-xylene-enriched extract 30 obtained from xylene separation zone 800. The reaction of para-xylene-depleted raffinate 26, comprising predominantly ortho-xylene, meta-xylene, and ethylbenzene, in isomerization reaction zone 700 is beneficial in terms of producing additional para-xylene, by promoting the reactions in this zone directionally toward achieving equilibrium levels of the xylene isomers in isomerization effluent 24. Representative embodiments of the invention therefore comprise separating, in a xylene separation zone, isomers of C₈ aromatic hydrocarbons in transalkylation product 23, into para-xylene-depleted raffinate 26 and para-xylene-enriched extract 30, the latter comprising essentially the overall amount of para-xylene, and generally the overall amount of C₈ aromatic hydrocarbons, obtained from the aromatics complex.

Isomerization reaction zone 700 generally comprises a catalyst, and is maintained under isomerization conditions, known for carrying out the desired isomerization and optionally dealkylation reactions, as discussed below. Typical isomerization conditions include a temperature from about 0° C. (32° F.) to about 600° C. (1112° F.), an absolute pressure from about 100 kPa (14.5 psi) to about 6 MPa (870 psi), and a WHSV (based on the isomerization combined feed) from about 0.1 hr⁻¹ to about 30 hr⁻¹. Hydrogen may be introduced, generally in a gaseous mixture (e.g., containing recycle hydrogen gas) at varying purity levels, to the isomerization reaction zone at a hydrogen-to-hydrocarbon molar ratio (based on the isomerization combined feed) of generally from about 0.5:1 to about 15:1, and typically from about 0.5:1 to about 10:1. In the case of maintaining liquid phase conditions in the isomerization reaction zone, typically no hydrogen is added.

Representative isomerization catalysts used in the isomerization reaction zone comprise a metal component and a molecular sieve component that may be zeolitic or non-zeolitic, in addition to an inorganic oxide component. The molecular sieve component may be selected according to the desired extent of ethylbenzene dealkylation (to benzene and light hydrocarbons) versus ethylbenzene isomerization (to additional xylenes), which generally depends on the overall demand for benzene. In general, zeolitic aluminosilicate molecular sieves are relatively more acidic and promote a greater degree of dealkylation (cracking) compared to non-zeolitic molecular sieves. Representative zeolitic aluminosilicate molecular sieves include pentasil zeolites, such as those having the structure types of MFI, MEL, MTW, MFS, MTF and FER (IUPAC Commission on Zeolite Nomenclature), MWW, beta zeolite, or mordenite. Representative non-zeolitic molecular sieves include those having one or more of the AEL framework types, for example SAPO-11, or one or more of the ATO framework types, for example MAPSO-31 (“Atlas of Zeolite Structure Types,” Butterworth-Heineman, Boston, Mass., 3^{rd} ed., 1992).

Representative metal components of isomerization catalysts include at least one noble metal and optionally at least one base metal modifier in addition to, or in place of, the at least one noble metal. Noble metals include platinum group metals selected from the group consisting of platinum, palladium, rhodium, ruthenium, osmium, iridium, and mixtures thereof. Base metals may be selected from the group consisting of rhenium, tin, germanium, lead, iron, cobalt, nickel, indium, gallium, zinc, uranium, dysprosium, thallium, and mixtures thereof. The metal component may also comprise combinations of one or more base metals and/or one or more noble metals. The total content of metal(s) in the isomerization catalyst is generally from about 0.01% to about 10% by weight, and typically from about 0.01% to about 3% by weight. The total content of the molecular sieve in the isomerization catalyst is generally from about 1% to about 99% by weight, typically from about 10% to about 90% by weight, and often from about 25% to about 75% by weight. Additional components of the isomerization catalyst may include an inorganic oxide component, such as binder material (e.g., alumina). It may be desirable to modify the isomerization catalyst by sulfiding, either in situ or ex situ. A representative isomerization catalyst for use in the isomerization reaction zone is described in U.S. Pat. No. 4,899,012, the teachings of which, with respect to isomerization catalyst formulations, are incorporated herein by reference.

The isomerization reaction zone may comprise one or more individual isomerization reactors containing isomerization catalyst in various catalyst bed configurations (e.g., fixed bed or moving bed) and flow configurations (e.g., axial flow or radial flow). Each of the one or more individual reactors may contain one or more types of isomerization catalyst. If two or more types are used, they may be blended, at varying blend ratios, or segregated in the individual reactor(s).

Representative embodiments comprise fractionating the isomerization effluent (exiting the isomerization reaction zone) to provide an isomerate product comprising the majority or essentially all of the trimethylbenzene and tetramethylbenzene produced in isomerization reaction zone 700, in addition to the majority or essentially all of the xylenes produced from isomerization of ethylbenzene in this zone. Associated with certain aspects of the invention, the production of these desired C₉ and C₁₀ methylated aromatic hydrocarbons, by the isomerization of aromatic hydrocarbons that are alkylated with higher alkyl groups, generally proceeds through the formation of naphthenic hydrocarbons (i.e., naphthenes or saturated ring hydrocarbons). The dehydrogenation of any such intermediates present in the isomerization effluent 24 is therefore desirable in terms of decreasing losses of aromatic hydrocarbons to their less valuable, corresponding naphthenes (naphthenic hydrocarbons). Moreover, these naphthenes, which may be present in the isomerization effluent in an amount ranging from 2-20% by weight, have boiling points that are similar to aromatic hydrocarbons and thereby interfere with their processing (e.g., transalkylation and separation) as described herein. For example, C₉ naphthenic hydrocarbons generally co-boil with xylenes and are therefore passed to the xylene separation zone (e.g., following fractionation of the isomerization effluent in the isomerate splitter and the fractionation of the bottoms fraction, exiting this splitter, in the xylene column to provide the xylene column overhead fraction containing the C₉ naphthenic hydrocarbons), before being recycled to the isomerization reaction zone. Also, C₁₀ naphthenic hydrocarbons generally co-boil with C₉ aromatic hydrocarbons and are therefore passed to the transalkylation reaction zone (e.g., following fractionation of the isomerization effluent in the isomerate splitter, the fractionation of the bottoms fraction, exiting this splitter, in the xylene column, and the further fractionation of the bottoms fraction, exiting this xylene column, in the A9 divided wall distillation column to provide the side cut fraction exiting this column and containing the C₁₀ naphthenic hydrocarbons). Additionally, C₈ naphthenic hydrocarbons tend to remain in the isomerization reaction zone recycle loop, which includes the xylene separation zone, in much the same manner as described above with respect to the C₉ naphthenic hydrocarbons. The formation of naphthenic hydrocarbons in isomerization zone 700 can therefore detrimentally lead to increased equipment and utility requirements, and/or loss of processing capacity, in a number of unit operations in an overall aromatics complex.

Such disadvantages may be reduced or even eliminated using a dehydrogenation reaction zone for converting naphthenes in the isomerization effluent to their corresponding aromatic hydrocarbons (e.g., by converting saturated rings to aromatic rings). Representative conversion levels of the total naphthenes in the isomerization effluent are generally from about 30% to 100%, typically from about 75% to about 100%, and often from about 90% to about 100%. A further advantage associated with the conversion of naphthenes in the isomerization effluent is that this permits the isomerization reaction zone to operate at a relatively high naphthene concentration, thereby increasing the rate of reaction of ethyl-, propyl-, and butyl-substituted aromatic hydrocarbons to the desired methylated aromatic hydrocarbons. Without being bound by theory, reactions in the isomerization reaction zone proceed initially through the generation of naphthenes at a high reaction rate at the beginning of this zone. In terms of the benefits of allowing a substantial concentration of naphthenes in the isomerization reaction zone, it is noted an increase in the C₈ naphthene concentration from 6% to 20% by weight in the isomerization combined feed generally increases the conversion (isomerization) of ethylbenzene to xylenes from 30% to 40% in the isomerization reaction zone.

The dehydrogenation reaction zone, if used, can be in the same reactor as the isomerization reaction zone (e.g., as a separate bed of catalyst downstream of a bed of isomerization catalyst), or in at least one of the reactors used in the isomerization reaction zone, in the case of multiple isomerization reactors. Otherwise, the dehydrogenation reaction zone can be in one or more separate reactors, or partially in one or more reactors of the isomerization reaction zone and partially in one or more separate reactors. According to one embodiment, the isomerization effluent is heated, for example, by about 28° C. to about 111° C. (about 50° F. to about 200° F.), to a temperature representative of dehydrogenation zone conditions, for example often in the range from about 315° C. (600° F.) to about 482° C. (900° F.). Representative conditions in the dehydrogenation reaction zone also include ranges of pressure and WHSV as discussed above with respect to the isomerization reaction zone.

Representative dehydrogenation catalysts used in the dehydrogenation reaction zone comprise a metal component and a molecular sieve component that may be zeolitic or non-zeolitic, in addition to an inorganic oxide component. Representative metal components, zeolitic and non-zeolitic molecular sieves, and inorganic oxide components (e.g., binder materials such as alumina), are as discussed above with respect to isomerization catalysts. A preferred metal component of the dehydrogenation catalyst comprises platinum. The total content of metal(s) in the dehydrogenation catalyst is generally from about 0.01% to about 10% by weight, typically from about 0.01% to about 3% by weight. The total content of the molecular sieve in the dehydrogenation catalyst is generally from about 1% to about 99% by weight, typically from about 10% to about 90% by weight, and often from about 25% to about 75% by weight.

Preferably, the molecular sieve component of the dehydrogenation catalyst is non-acidic, such that the desired naphthene ring dehydrogenation occurs with minimal cracking, ring opening, and dealkylation side reactions, all of which decrease the yield of the desired alkyl aromatic (e.g., methylated aromatic) hydrocarbons. According to representative embodiments, the acidity of the dehydrogenation catalyst is less than the acidity of the isomerization catalyst. If a catalyst blend is used in either or both of the isomerization reaction zone and dehydrogenation reaction zone, then the acidity of the blend (and consequently the acidity of the catalyst used in particular zone) is based on a weighted average value of acidity of the individual catalysts of the blend. Acidity may be determined, for example in units of moles of acid sites per gram of catalyst, by temperature programmed desorption (TPD) of a quantity of ammonia, from an ammonia-saturated sample of the catalyst, over a temperature from 275° C. (527° F.) to 500° C. (932° F.), which is beyond the temperature at which the ammonia is physisorbed. The quantity of acid sites therefore corresponds to the number of moles of ammonia that is desorbed in this temperature range.

In the particular embodiment illustrated in the FIGURE, isomerization effluent 24 is passed to dehydrogenation reaction zone 750 to dehydrogenate naphthenes to their corresponding aromatic hydrocarbons as discussed above. Representative methods therefore comprise dehydrogenating, in the dehydrogenation reaction zone, C₉ and C₁₀ naphthenic hydrocarbons in the isomerization effluent, thereby providing a dehydrogenation effluent that is depleted in these hydrocarbons, relative to the isomerization effluent. Dehydrogenation effluent 27 (exiting dehydrogenation reaction zone 750) is fractionated in isomerate splitter 900 to provide a high boiling fraction (e.g., a bottoms product), such as isomerate product 32 comprising all or substantially all (e.g., 99% by weight or more) of the trimethylbenzene and tetramethylbenzene produced in isomerization reaction zone 700 and dehydrogenation reaction zone 750, as well as generally all or substantially all (e.g., 99% by weight or more) of the C₈ and higher carbon number aromatic hydrocarbons in dehydrogenation effluent 27. Isomerate splitter 900 additionally provides a low boiling fraction (e.g., overhead product), such as light ends product 34 comprising toluene and byproducts having a normal boiling point below that of toluene. Such byproducts include light byproducts (e.g., light alkane hydrocarbons) resulting from side reactions such as dealkylation in isomerization reaction zone 700 and/or dehydrogenation reaction zone 750. Isomerate product 32, transalkylation product 23, and a reformate splitter fraction, such as bottoms product 2 of a reformate splitter (not shown) may be fractionated in xylene column 100 to provide, as a fraction of these combined streams, a feed to xylene separation zone 800, which is namely xylene column overhead fraction 28 in the embodiment illustrated in the FIGURE.

Further aspects of the invention relate to methods for producing C₈ aromatic hydrocarbons. Representative methods comprise isomerizing, in an isomerization reaction zone, a methylated aromatic hydrocarbon-depleted fraction to provide an isomerization effluent comprising an additional amount of methylated aromatic hydrocarbons. These methods may further comprise fractionating the isomerization effluent (for example, using isomerate splitter 900, xylene column 100, A9 divided wall distillation column 500, and/or A10 divided wall distillation column 600, as illustrated in the FIGURE) to provide one or more methylated aromatic hydrocarbon-enriched fractions. The methods may further comprise reacting the one or more methylated aromatic hydrocarbon-enriched fractions in a transalkylation reaction zone to provide a transalkylation effluent comprising the C₈ aromatic hydrocarbons. According to particular embodiments, the methylated aromatic hydrocarbon-depleted fraction and a para-xylene-depleted raffinate of a xylene separation zone are isomerized in the isomerization reaction zone. According to other particular embodiments, (i) a trimethylbenzene-depleted fraction obtained from fractionating an aromatic hydrocarbon-containing stream comprising C₉ and C₁₀ aromatic hydrocarbons and/or (ii) a tetramethylbenzene-depleted fraction obtained from fractionating an aromatic hydrocarbon-containing stream comprising C₁₀ aromatic hydrocarbons, are isomerized in the isomerization reaction zone. According to yet other embodiments, after isomerization and before fractionation of the isomerization effluent, the methods may further comprise dehydrogenating, in a dehydrogenation reaction zone, C₉ and C₁₀ naphthenic hydrocarbons in the isomerization effluent.

Overall aspects of the invention relate to methods for producing C₈ aromatic hydrocarbons, comprising reacting a methylated aromatic hydrocarbon in a transalkylation reaction zone to provide a transalkylation effluent comprising the C₈ aromatic hydrocarbons. Advantageously, the methylated aromatic hydrocarbon is present in a methylated aromatic hydrocarbon-enriched fraction to the transalkylation reaction zone. Those having skill in the art, with the knowledge gained from the present disclosure, will recognize that various changes could be made in these C₈ aromatic hydrocarbon production methods without departing from the scope of the present invention. To the extent that the methods described herein involve steps of processing (e.g., “fractionating,” “reacting,” “transalkylating,” “isomerizing,” “dehydrogenating,” etc.) various feeds, products, and fractions (e.g., “feed stream,” “methylated aromatic hydrocarbon-enriched fraction,” “transalkylation effluent,” “isomerate product,” etc.), it is understood that such steps also include processing portions of these feeds, products, and fractions (e.g., the majority of these feeds, products, and fractions), as portions, such as minor amounts, are often removed at various points for reducing equipment loading, prevention of byproduct accumulation, sampling, etc. Mechanisms used to explain theoretical or observed phenomena or results, shall be interpreted as illustrative only and not limiting in any way the scope of the appended claims.

1. A method for producing C₈ aromatic hydrocarbons, the method comprising:
(a) fractionating a feed stream comprising C₉ or C₁₀ aromatic hydrocarbons, to provide a methylated aromatic hydrocarbon-enriched fraction; and
(b) reacting the methylated aromatic hydrocarbon-enriched fraction in a transalkylation reaction zone to provide a transalkylation effluent comprising the C₈ aromatic hydrocarbons.

2. The method of claim 1, wherein the methylated aromatic hydrocarbon-enriched fraction contains a higher percentage by weight of methylated aromatic hydrocarbons, based on aromatic hydrocarbons of the same carbon number, relative to the feed stream.

3. The method of claim 2, wherein the methylated aromatic hydrocarbon-enriched fraction is a trimethylbenzene-enriched fraction obtained from fractionating the feed stream, and wherein the feed stream comprises C₉ and C₁₀ aromatic hydrocarbons.

4. The method of claim 3, wherein the trimethylbenzene-enriched fraction is obtained as a side cut product of an A9 divided wall distillation column for fractionating the aromatic hydrocarbon-containing feed stream.

5. The method of claim 4, further comprising fractionating a C₁₀ aromatic hydrocarbon-enriched fraction of the A9 divided wall distillation column, to provide a tetramethylbenzene-enriched fraction.

6. The method of claim 2, wherein the methylated aromatic-enriched fraction is a tetramethylbenzene-enriched fraction obtained from fractionating a feed stream, and wherein the feed stream comprises C₁₀ aromatic hydrocarbons.

7. The method of claim 6, wherein the tetramethylbenzene-enriched fraction is obtained as a side cut product of an A10 divided wall distillation column for fractionating the aromatic hydrocarbon-containing stream.

8. The method of claim 1, wherein step (a) comprises:
(a1) fractionating the feed stream comprising C₉ and C₁₀ aromatic hydrocarbons, to provide a trimethylbenzene-enriched fraction, a trimethylbenzene-depleted fraction, and a C₁₀ aromatic hydrocarbon-enriched fraction;
(a2) fractionating the C₁₀ aromatic hydrocarbon-enriched fraction to provide a tetramethylbenzene-enriched fraction, a tetramethylbenzene-depleted fraction, and a heavy aromatics fraction
and wherein step (b) comprises:
(b) reacting the trimethylbenzene-enriched fraction and the tetramethylbenzene-enriched fraction in the transalkylation reaction zone to provide the transalkylation effluent comprising the C₈ aromatic hydrocarbons.

9. The method of claim 8, further comprising:
(c) producing trimethylbenzene and tetramethylbenzene by isomerizing the trimethylbenzene-depleted fraction and the tetramethylbenzene-depleted fraction in an isomerization reaction zone to provide an isomerization effluent comprising the trimethylbenzene and tetramethylbenzene.

10. The method of claim 9, wherein the trimethylbenzene-depleted fraction, the tetramethylbenzene-depleted fraction, and a para-xylene-depleted raffinate from a xylene separation zone are reacted in the isomerization reaction zone.

11. The method of claim 9, further comprising dehydrogenating, in a dehydrogenation reaction zone, C₉ and C₁₀ naphthenic hydrocarbons in the isomerization effluent.

12. The method of claim 9, further comprising fractionating the isomerization effluent to provide an isomerate product comprising the trimethylbenzene and the tetramethylbenzene.

13. The method of claim 12, further comprising fractionating the transalkylation effluent to provide a transalkylation product fraction comprising the C₈ aromatic hydrocarbons.

14. The method of claim 13, wherein the transalkylation product fraction is recovered as a high boiling fraction from a toluene column that provides a toluene-enriched product as a low boiling fraction, the method further comprising partly or completely recycling the toluene-enriched product to the transalkylation zone.

15. The method of claim 1, further comprising separating, in a xylene separation zone, isomers of the C₈ aromatic hydrocarbons into a para-xylene-depleted raffinate.

16. A method for producing C₈ aromatic hydrocarbons, the method comprising:
(a) isomerizing, in an isomerization reaction zone, a methylated aromatic hydrocarbon-depleted fraction to provide an isomerization effluent comprising an additional amount of methylated aromatic hydrocarbons;
(b) fractionating the isomerization effluent to provide a methylated aromatic hydrocarbon-enriched fraction; and
(b) reacting the methylated aromatic hydrocarbon-enriched fraction in a transalkylation reaction zone to provide a transalkylation effluent comprising the C₈ aromatic hydrocarbons.

17. The method of claim 16, wherein, in step (a), the methylated aromatic hydrocarbon-depleted fraction and a para-xylene-depleted raffinate of a xylene separation zone are isomerized in the isomerization reaction zone.

18. The method of claim 16, wherein step (a) comprises isomerizing, in the isomerization reaction zone, a trimethylbenzene-depleted fraction obtained from fractionating an aromatic hydrocarbon-containing stream comprising C₉ and C₁₀ aromatic hydrocarbons and a tetramethylbenzene-depleted fraction obtained from fractionating an aromatic hydrocarbon-containing stream comprising C₁₀ aromatic hydrocarbons.

19. The method of claim 16, further comprising, after step (a) and before step (b), dehydrogenating, in a dehydrogenation reaction zone, C₉ and C₁₀ naphthenic hydrocarbons in the isomerization effluent.

20. A method for producing C₈ aromatic hydrocarbons, the method comprising reacting a methylated aromatic hydrocarbon in a transalkylation reaction zone to provide a transalkylation effluent comprising the C₈ aromatic hydrocarbons, wherein the methylated aromatic hydrocarbon is present in a methylated aromatic hydrocarbon-enriched fraction to the transalkylation reaction zone.
